(19) United States
(12) Patent Application Publication
(10) Pub. No.: US 2026/0265291 A1
(43) Pub. Date: Sep. 10, 2026

(54) USE OF MOLECULES ISOLATED FROM BACTERIA PRESENT IN DIGESTATE PRODUCED BY BIOGAS PLANTS AS IMMUNOSTIMULANTS OF PLANT DEFENCE RESPONSES

(71) Applicants: UNIVERSITA' DEGLI STUDI DI ROMA “LA SAPIENZA”, Roma (IT); AGROLIO SRL, Andria (IT)

(72) Inventors: Vincenzo LIONETTI, Roma (IT); Savino AGRESTI, Andria (IT); Giulia DE LORENZO, Roma (IT); Marco GRECO, Roma (IT)

(21) Appl. No.: 19/162,490

(22) PCT Filed: Mar. 8, 2024
(86) PCT No.: PCT/EP2024/056272

§ 371 Date: Sep. 5, 2025

(30) Foreign Application Priority Data
Mar. 14, 2023 (IT) 102023000004800

(51) Int. Cl. C07K 1/02; A01N 63/20; A01N 63/50; A01P 21/00; C07K 1/14; C07K 14/195; C07K 14/36

(52) U.S. Cl. CPC C07K 1/02; A01N 63/20; A01N 63/50; A01P 21/00; C07K 1/14; C07K 14/195; C07K 14/36

(57) ABSTRACT

A process is described for the valorisation of the microbial fraction contained in the waste material from biogas plants following anaerobic digestion of olive pomace, thanks to which an extract enriched in proteins and peptides of microbial origin is obtained for use as a plant protection product in agricultural production.

### BACKGROUND OF THE INVENTION

The present invention relates to the fields of agriculture, biology, plant physiology, plant pathology, biopesticides, agro-industry, biogas, floriculture and nursery gardening, in that it describes a process for recovering waste materials from biogas plants, from which a product is obtained in the form of an extract enriched in molecules of microbial origin to be used for phytosanitary treatment and the improvement of the quality and yield of agricultural production.

### BACKGROUND ART

A serious risk to the well-being of the environment is posed by chemical pesticides, which are widely used today to treat plant diseases, but are highly damaging to health and extremely expensive. Among the environmentally-sustainable methods of disease control is the possibility of stimulating the plants' immune response before the attack by the pathogenic organism. Immunostimulation is possible thanks to the use of natural compounds, elicitors of defence responses to counter plant infection by phytopathogens (Mauch-Mani et al., 2017; Rizvi et al., 2009).

In nature, plants have the ability to recognise bacteria by recognising certain microbial molecules which are perceived by specific plant receptors as alarm signals and which trigger natural self-defence mechanisms. These molecules belong to the class of pathogen associated molecular patterns (PAMPs). Several bacterial PAMPs, including peptides and lipopolysaccharides (LPS), are known as elicitors of defence responses (Albert, 2013).

The most studied include flagellin fragments (flg22 and flgII-28), a protein of the bacterial flagellum; a cell lining protein called Hairpin (in the form of a hairpin) (Choi et al., 2013) and the 18-amino acid peptide contained in the elongation factor Tu (EF-Tu), called elf18, which is one of the most abundant and conserved proteins in the prokaryotic kingdom (Yamamoto et al., 2014). By binding to various receptors located in the plant cell membrane, PAMPs induce a very rapid and intense biochemical alarm signal, which spreads in the plant and ‘switches on’ various physiological defence responses, triggering a dedicated immunity (known as Pattern Triggered Immunity—PTI) (Yuan, M., Ngou, B. P. M., Ding, P., Xin, X.-F., 2021. PTI-ETI crosstalk: an integrative view of plant immunity. Current Opinion in Plant Biology, Biotic interactions 62, 102030. https://doi.org/10.1016/j.pbi.2021.102030).

Some examples of commercial natural immune-stimulating products are known:
trade name COS-OGAR (FytoFend SA Belgium), authorised in Europe (Reg. EU 543/2015), consisting of a complex of natural origin containing chito-oligosaccharides derived from the (COS) depolymerisation of chitosan, extracted from the chitin of the exoskeleton of crustaceans, and oligogalacturonides (OGA) derived from the degradation of pectins extracted from the peel of citrus fruits and apples, capable of enhancing the defence responses of vines against powdery mildew caused by Uncinula necator and of potatoes against Phytophthora infestans, the causal agent of Downy mildew;
trade name Regalia® (Marrone Bio Innovations USA) is a formulation consisting of plant extracts with elicitor activity derived from Reynoutria sachalinensis (common name Sachalin polygonus), used to preventively combat powdery mildew, blight, anthracnose and other bacterial diseases;
trade name Messenger®, formulated based on Hairpin protein, effective on a wide variety of economically important crops, such as cotton, wheat, cucumber, citrus, tobacco, strawberry, tomato and peppers against viruses, nematodes and fungi.

Cerda, A., Mejias, L., Rodriguez, P., Rodríguez, A., Artola, A., Font, X., Gea, T., Shchez, A., 2019. Valorisation of digestate from biowaste through solid-state fermentation to obtain value added, bioproducts: A first approach. Bioresource Technology 271, 409-416. https://doi.org/10.1016/j.biortech.2018.09.131 describes a digestate as a possible growth substrate for Bacillus thuringiensis, capable of producing a toxin to be used as a biopesticide for insects.

US patent application, publication no. US20140024529, describes the use of the nitrogen component and soil conditioner capacity of an algae-derived digestate as a fertiliser capable of improving plant growth and health.

Chinese patent no. CN102503660 describes the use of a mixture with a small percentage of a phytoregulator called sodium nitrophenolate and dispersants, antifreeze, additives and fillers as manure, fertiliser, and to improve plant disease resistance.

### Closest Prior Art to the Invention

Newman M. A., et al., ‘MAMP (microbe-associated molecular pattern) triggered immunity in plants’, Frontiers in Plant Science, vol. 4, 2013 Jan. 1, is a review summarising the findings on several microbial molecules (defined as MAMPs), such as EF-Tu, peptidoglycan, lipopolysaccharide, fungal chitin and flagellin which plants are capable of sensing through specific receptors, activating defence responses.

European Patent no. EP2967052 describes ascarosides, pheromones secreted by nematodes or of synthetic origin, as inducers of plant defence responses against pathogens.

Mishina T. E. et al., “Pathogen-associated molecular pattern recognition rather than development of tissue necrosis contributes to bacterial induction of systemic acquired resistance in Arabidopsis”, The Plant Journal, vol. 50, no. 3, 2007 Apr. 5, 500-513, describes the defence responses in Arabidopsis thaliana induced by treatment with the bacterium Pseudomonas syringae and PAMP molecules such as flagellin and lipopolysaccharides.

International patent application no. WO2023/038969 describes a composition to enhance the plant immune response where non-pathogenic microorganisms are used which can be formulated as a dustable powder and the protection method is based on contacting the plant or the soil in which it grows with elicitors such as flagellin peptides, peptidoglycans and lipopolysaccharides.

European patent no. EP4011854 describes the use of the mineral component in liquid digestate from biogas plants fed with waste from olive oil production as a fertiliser or Lolium growth regulator.

### Technical Problem

One of the most important problems in the agro-food industry is waste management. Considered until recently as waste, they were dumped into the environment with serious repercussions on agriculture and the health of natural ecosystems. In a circular economy perspective, several companies reuse waste biomass from their primary agro-food production as feed for a biogas biodigester in a secondary industrial anaerobic digestion process aimed at the production of renewable energies such as biogas and thermoelectric energy.

Recently, also stimulated by the National Recovery and Resilience Plan (NRRP) (Ministry of Ecological Transition, Decree of 5 Aug. 2022, https://www.gazzettaufficiale.it/eli/id/2022/08/18/22A04725/sg), several municipalities have been investing in the construction of biodigesters to stabilise the organic biomass of city waste and convert it into bioenergy in the form of biogas. According to the European Biogas Association (EBA) and the Italian Biogas Consortium (CIB), there are now more than 1,500 biogas plants in Italy (second in Europe only to Germany).

Biogas is mainly composed of methane and carbon dioxide and is the result of the microbial degradation of organic matter such as livestock effluents, waste or dedicated plant biomass, animal by-products, sewage sludge and the organic fraction of municipal solid waste through the four biochemical steps of microbial anaerobic digestion: hydrolysis, acidogenesis, acetogenesis and methanogenesis. As a result of this industrial bioconversion, large quantities of a waste known as digestate are also produced. The resulting digestate is presented as a semi-solid residue, where the solid part is mainly represented by partially degraded residual organic matter and the liquid part is mainly composed of a high content of inorganic compounds such as nitrogen (N), phosphorus (P), potassium (K) sulphur(S) and various micronutrients. Furthermore, the digestate is highly enriched with different populations of microbes responsible for the anaerobic digestion of plant biomass. The most frequently identified species in the digestate belong to the phyla Firmicutes, Bacteroides and Spirochetes, which in nature live in extreme environments such as biothermal springs, gastrointestinal tracts and saline environments.

In order to encourage an ecological transition which eliminates chemical synthesis products in favour of the use of natural substances, the Italian government recently equated digestate to a by-product to be used as a fertiliser in place of synthetic chemical fertilisers among the urgent measures to counter the economic and humanitarian effects of the Ukrainian crisis (Italian Decree-Law no. 21 of 21 Mar. 2022). However, important controls are required in this transition so that the quality guarantees of the supply chain are respected. However, to date, digestate still represents poorly and little re-used industrial waste, causing a management problem for companies. Digestate can be dried and burnt, but with the release of harmful carbon gases into the air. Digestate has the disadvantage of being very wet, complicating the transport and distribution of the nutrients it contains. Furthermore, its release into the environment can cause various problems such as the emission of ammonia and greenhouse gases, nutrient leaching and the transfer of pathogens which may have been selected in the production process and micropollutants (Nkoa, R., 2014. Agricultural benefits and environmental risks of soil fertilization with anaerobic digestates: a review. Agron. Sustain. Std. 34, 473-492. https://doi.org/10.1007/s13593-013-0196-z).

The absence of efficient strategies for sustainable digestate management could cripple the biogas plant industry. Important research efforts are still needed to valorise digestate with treatments which lead to the production of high value-added products.

Adding to this environmental problem is the massive use of chemical pesticides which are currently necessary to control plant diseases but have negative effects on human health and the spread of plant diseases. Some pathogens can survive exposure to pesticides thanks to the occurrence of genetic mutations arising from pesticide exposure which cause the onset of resistance to the active ingredient (Nicolopoulou-Stamati, P., Maipas, S., Kotampasi, C., Stamatis, P., Hens, L., 2016. Chemical Pesticides and Human Health: The Urgent Need for a New Concept in Agriculture. Frontiers in Public Health 4; Gould, F., Brown, Z. S., Kuzma, J., 2018. Wicked evolution: Can we address the sociobiological dilemma of pesticide resistance? Science 360, 728-732. https://doi.org/10.1126/science.aar3780).

Therefore, there is an urgent need for new solutions for plant protection in agriculture. One sustainable possibility is the stimulation of the plants' immune response with natural substances which protect them from the attack of pathogenic organisms. The commercial products already available are formulations of molecules obtained through recombinant technologies such as heterologous peptide expression, which require high production costs and specific laboratories and equipment such as bioreactors.

In the light of the above, it is evident how strongly felt the need is in the field of the present invention to find a solution for the valorisation of digestate from biogas production, especially its microbial component, and a sustainable alternative to the use of chemical pesticides in plant disease control.

In this sense, the inventors have devised a process aimed at valorising the microbial fraction present in digestate so as to obtain a product for use as a plant immunity biostimulant. The extract obtained from the proposed process contains a mixture of proteins and peptides of microbial origin obtained from digestate from the anaerobic digestion of olive pomace which can pre-stimulate the immune system of plant crops, making them more ready to resist phytopathogenic microorganisms and thus more productive and of higher quality. Digestate waste is thus re-evaluated as a bioresource for the isolation of molecules useful in the production of a low-cost ‘green’ agrochemical and biostimulant which represents an environmentally-sustainable alternative to chemical pesticides, pursuing a model of circular economy and sustainable agriculture.

The invention therefore finds application in environmentally-friendly agribusiness, floriculture and nursery processes and in the proliferation of eco-innovative sectors based on green technologies contributing to economic and environmental sustainability. The proposed process also means that the digestate used is deprived of the microbial fraction, which is completely inactivated and loses its potentially polluting capacity. This process thus solves a potentially environmentally hazardous and as yet unaddressed problem of the dispersion of undefined bacterial populations present in digestate into agricultural soils when used as fertiliser.

With respect to the known art closest to invention, Newman M. A., et al. 2013 reviewed several scientific papers indicating MAMPs as stimulators of defence responses in plants. The molecules described by Newman are not isolated from the microbial fraction of digestate from the anaerobic digestion of olive pomace, as described by this invention.

The present invention also differs from European patent EP2967052. The molecules covered by this patent are ascarosides, composed of a deoxy-sugar (called ascarolyse) linked to fatty acid side-chains, either of synthetic origin or from nematodes, small vermiform animals which nestle in the roots of plants and prevent them from developing. These molecules, as well as nematodes, are absent in the digestate.

Mishina T. E. et al. 2007 uses the flg22 peptide corresponding to the active domain demonstrated for bacterial flagellin chemically synthesised in the work by Mimotopes (www.mimotopes.com). The lipopolysaccharide preparation comes from bacterial cultures in the laboratory and purchased was from Sigma-Aldrich (http://www.sigmaaldrich.com/). It does not relate to a process of converting digestate into plant immunostimulants.

International patent WO2023/038969 considers elicitor molecules derived from a fungus antagonistic to other potentially pathogenic fungi such as Thricoderma harzianum and a rhizosphere bacterium such as Bacillus amyloliquefaciens in fermentative culture. These microorganisms are not present in the digestate from anaerobic digestion aimed at biogas production.

Patent EP4011854 valorises the mineral component present in the liquid digestate obtained from the anaerobic digestion of waste from olive oil production, as a fertiliser or Lolium growth regulator. The process differs from that of the present invention because it does not involve isolation of the microbial component, nor sonication. Fertilising power is associated with mineral components such as nitrogen and potassium, nutrients useful for plant growth and not identified as defence response elicitors. The mineral component of the liquid fraction of the digestate is not present in the protein mixture of the present invention, but rather is removed during the preparation process.

### OBJECT OF THE INVENTION

The above technical problem is solved by providing a process for the treatment of digestate derived from anaerobic digestion of olive pomace, wherein the process comprises the following steps:
a) sedimentation of the digestate obtained from the anaerobic digestion of olive pomace to obtain a liquid fraction and a solid fraction;
b) sampling the liquid fraction obtained at the end of step a) and centrifugation thereof to obtain a liquid supernatant fraction and a solid-pellet fraction, wherein said solid-pellet is of bacterial origin;
c) resuspension of the solid-pellet of bacterial origin obtained at the end of step b);
d) mechanical cellular breaking by sonication of the microbes in the suspension obtained at the end of step c);
e) further centrifugation of the solution obtained at the end of step d) to obtain a cellular residue fraction and a liquid phase constituting the final product;
   wherein the final product is a non-infectious solution comprising a PAMPS fraction enriched in proteins and peptides of bacterial origin (also referred to as PAMPs fraction), wherein optionally said PAMPs fraction further contains lipopolysaccharides with PAMPs activity, derived from the bacterial membrane and solubilised during the sonication.

Further subject matter of the present invention is the final product, i.e., a non-infectious, bacterial-protein and bacterial-peptides enriched solution comprising a PAMPS fraction, wherein optionally said PAMPs fraction further contains lipopolysaccharides with PAMPs activity, derived from the bacterial membrane and solubilised during the sonication.

Still another object of the present invention is a non-infectious, bacterial-protein and bacterial-peptides enriched solution comprising a PAMPS fraction, wherein optionally said PAMPs fraction further contains lipopolysaccharides with PAMPs activity, derived from the bacterial membrane and solubilised during the sonication, obtained by the process comprising the following steps:
a) sedimentation of the digestate obtained from the anaerobic digestion of olive pomace to obtain a liquid fraction and a solid fraction;
b) sampling the liquid fraction obtained at the end of step a) and centrifugation thereof to obtain a liquid supernatant fraction and a solid-pellet fraction, wherein said solid-pellet is of bacterial origin;
c) resuspension of the solid-pellet of bacterial origin obtained at the end of step b);
d) subjecting the resuspended solid-pellet of bacterial origin obtained at the end of step c) to mechanical cellular breaking by sonication to obtain a solution;
e) subjecting the solution obtained at the end of step d) to further centrifugation to obtain a cellular residue fraction and a liquid phase constituting the final product;

It is further an object of the present invention to use the aforesaid non-infectious, bacterial-protein and bacterial-peptides enriched solution comprising a PAMPS fraction and further containing lipopolysaccharides with activity as an immunostimulant for the plant immune system.

Further characteristics of the present invention will be clear from the following detailed description, with reference to experimental data reported and the appended figures.

### BRIEF DESCRIPTION OF THE FIGURES

FIG. 1 schematically illustrates the preparation steps of the PAMPs fraction.

FIG. 2 graphically shows that the PAMPs fraction induces the expression of defence genes in Arabidopsis seedlings grown in liquid medium. The expression of CYP81F2 and FRK1 was assessed in seedlings treated with water (H₂O) and PAMPs fraction by means of qRT-PCR. The expression levels were normalised with respect to the expression of Ubiquitin 5.

FIG. 3 graphically shows the effect of treating Arabidopsis seedlings with different concentrations of PAMPs fraction. The concentration values are expressed in micrograms per millilitre (μg/ml) and seedling weight in milligrams (mg). The bars indicate the mean±standard deviation (n=6).

FIG. 4 shows that the PAMPs fraction acts as an immunostimulant and protector of Arabidopsis against infection by the necrotrophic fungus B. cinerea. Leaves of adult plants were pre-treated with PAMPs fraction and water (H₂O) and 24 h after pretreatment were infected with B. cinerea. A and B, CYP81F2 and PAD3 gene expression was quantified by means of qRT-PCR 8 h after infection with Botrytis. The expression levels were normalised with respect to the expression of Ubiquitin 5. C, Photographs of leaves showing symptoms of infection by the fungus B. cinerea. D, Quantification of the lesion area of Arabidopsis leaves 48 h after infection. Mock=leaves pre-treated with water and inoculated with only the fungus growth medium. The bars indicate mean±standard error (n=6). The asterisks indicate a statistically significant difference with respect to the treatment with water (H₂O) according to Student's t-test (p<0.001).

### DETAILED DESCRIPTION OF THE INVENTION

Within the meaning of the present invention, waste material from biogas production means a complex matrix containing partially degraded organic matter, inorganic compounds and microbial biomass, wherein the microbial biomass contains various microbial species, which in the context of the present invention is indicated as digestate.

Further, within the meaning of the present invention, digestate means the by-product obtained in company or inter-company plants from the anaerobic digestion, possibly also associated with other physical-mechanical treatments, of livestock manure or residues of vegetable origin or residues from the processing or valorisation of vegetable productions carried out by the agro-food industry, delivered as by-products, even if mixed together, used for agronomic purposes. Said by-product is defined by Art. 184bis of Italian Legislative Decree no. 152 of 3 Apr. 2006 where it is defined that a by-product and not a waste is any substance or object originating from a production process, of which it is an integral part, where the primary purpose of the production process is not the production of such a substance or object.

Within the meaning of the present invention, digestate is an intermediate by-product and not the final product obtained from the anaerobic digestion of olive pomace, which is a waste product from olive oil production plants.

Within the meaning of the present invention and as a definition known in scientific field, PAMPs (acronym for Pathogen Associated Molecular Patterns) refers to microbial molecules which are perceived by specific plant receptors as alarm signals and which induce natural physiological defence mechanisms.

Within the meaning of the present invention, PAMPs fraction means a mixture enriched in proteins and peptides of bacterial origin obtained from waste materials from agro-industrial processes involving anaerobic digestions of biomass and characterised by having no bacterial load and no infectious capacity, while exhibiting PAMPs activity.

Within the meaning of the present invention, the PAMPs fraction is a non-infectious mixture enriched in proteins and peptides of bacterial origin obtained by means of specific treatment of the liquid portion of the digestate from the anaerobic digestion of olive pomace. Said PAMPs fraction is mainly protein but can also contain lipopolysaccharides (LPS) derived from the bacterial membrane solubilised during sonication, which also have PAMPS activity.

Thus, a process for the treatment of digestate from biogas plants is provided, comprising the following steps:
a) sedimentation of the digestate obtained from the anaerobic digestion of olive pomace to obtain a liquid fraction and a solid fraction;
b) sampling the liquid fraction obtained at the end of step a) and centrifugation thereof to obtain a liquid supernatant fraction and a solid-pellet fraction, wherein said solid-pellet is of bacterial origin;
c) resuspension of the solid-pellet of bacterial origin obtained at the end of step b);
d) subjecting the resuspended solid-pellet of bacterial origin obtained at the end of step c) to mechanical cellular breaking by sonication to obtain a solution;
e) subjecting the solution obtained at the end of step d) to further centrifugation to obtain a cellular residue fraction and a liquid phase constituting the final product;
wherein the end product is a non-infectious (devoid of infectious capacity) bacterial-protein and/or bacterial-peptides enriched solution, also referred to as PAMPs fraction, optionally further comprising lipopolysaccharides, with PAMPs activity, derived from the bacterial membrane and solubilised during sonication.

The final product is a non-infectious (devoid of infectious capacity), bacterial-protein and bacterial-peptides enriched solution, also referred to as PAMPs fraction optionally and further comprising lipopolysaccharides with PAMPs activity.

The lipopolysaccharides with PAMPs activity are derived from the bacterial membrane solubilised during sonication.

The non-infectious (devoid of infectious capacity), bacterial-protein and bacterial-peptides enriched solution comprising a PAMPS fraction, optionally further comprising lipopolysaccharides, with PAMP activity, is obtained by the process comprising the following steps:
a) sedimentation of the digestate obtained from the anaerobic digestion of olive pomace to obtain a liquid fraction and a solid fraction;
b) sampling the liquid fraction obtained at the end of step a) and centrifugation thereof to obtain a liquid supernatant fraction and a solid-pellet fraction, wherein said solid-pellet is of bacterial origin;
c) resuspension of the solid-pellet of bacterial origin obtained at the end of step b);
d) subjecting the resuspended solid-pellet of bacterial origin obtained at the end of step c) to mechanical cellular breaking by sonication to obtain a solution;
e) subjecting the solution obtained at the end of step d) to further centrifugation to obtain a cellular residue fraction and a liquid phase constituting the final product;

The solution comprising a non-infectious, bacterial-protein and bacterial-peptides enriched solution comprising a PAMPS fraction, wherein the PAMPs fraction can optionally further comprising lipopolysaccharides (LPS) with PAMPs activity (also referred to as the PAMPs fraction), is used as an immunostimulant for the plant immune system.

The non-infectious, bacterial-protein and bacterial-peptides enriched solution comprising a PAMPS fraction optionally further comprising lipopolysaccharides with PAMPs activity.

The digestate can take the form of a complex matrix comprising partially degraded organic matter, inorganic compounds and microbial biomass.

The microbial biomass preferably consisting of one or more bacterial species belonging to the phyla selected from the group consisting of: Acidobacteria, Actinobacteria, Bacteroides, Chloroflexi, Spirochetes, Euryarchaeota, Firmicutes, Proteobacteria, Thermotogae, Tenericutes and Verrucomicrobes.

Even more preferably, the microbial biomass consisting of one or more bacterial species belonging to the phyla of Firmicutes, Bacteroides and Spirochetes.

Preferably, the digestate comes from the production of biogas from olive oil pomace, specifically from the anaerobic digestion of olive pomace.

Preferably in step a), the sedimentation is carried out at a temperature comprised between 4° C. and 10° C. for a time comprised between 8 and 24 hours.

More preferably in step a), the sedimentation is carried out at 4° C. for 24 hours.

Preferably in step b), the centrifugation is carried out at a temperature comprised between 4° C. and 10° C.

Preferably in step b), the centrifugation is carried out at 4° C.

Preferably in step b), the centrifugation is carried out at a speed comprised between 4,000 and 8,000 rpm, more preferably 8,000 rpm.

Preferably in step b), the centrifugation is carried out for a time comprised between 15 and 5 minutes, preferably 5 minutes.

Preferably in step c), the pellet is resuspended in distilled water.

For greater stability and preservation of the protein component, the pellet can be resuspended in 1/50 buffer with 20 mM TrisHCl pH 7.4+500 mM NaCl+1/200 protease inhibitor or in 1/50 buffer with 50 mM TrisHCl pH 8.8, 100 mM NaCl, 5% (v/v) glycerol, 1 mM &-aminocaproic acid, 1 mM phenylmethylsulfonyl fluoride and 0.2M benzamidine.

Preferably in step d), the mechanical cellular breaking is conducted through sonication with sound waves at a frequency comprised between 20 kHz and 40 kHz with sonication conditions preferably comprised between 40% and 100% amplitude and a pulsing function (pulser) between 5 seconds—ON and 5 seconds—OFF and 20 seconds—ON and 20 seconds—OFF. Even more preferably, 70% amplitude and a 20-second—ON and 20-second—OFF pulser function. Or through a cycle to be repeated two or three times of freezing at temperatures between −20° C. and −80° C. or in liquid nitrogen and thawing at +4° C. or at 6-10° C. in a thermostatic bath for 10 min and cooling to +4° C. or by rapid stirring in the presence of small glass beads with a diameter of 0.1 mm conducted in two 3-minute intervals in a Bead Beater or Mixer mill, separated by a 3-minute cooling period in ice.

Preferably in step e), the centrifugation is carried out at a temperature comprised between 4° C. and 10° C., at a speed comprised between 10,000 and 15,000 rpm for a time comprised between 15 and 50 minutes. Even more preferably, at 4° C. at 15,000 rpm for 30 minutes.

Preferably in the solution comprising a PAMPS protein fraction of bacterial origin, the protein fraction is present in an amount comprised between 10 and 500 μg/ml, more preferably the protein fraction is present in a concentration of 173.8 μg/ml.

### Examples

### Materials and Methods

Microbial DNA extraction, sequencing by Next Generation Sequencing (NGS) of 16S rRNA, and bioinformatic analysis of the sequencing results were carried out as previously described (Agostini et al., 2023).

Presence of protein in the PAMPs fraction was assessed by means of SDS-PAGE and gel staining with Coomassie Brilliant Blue dye as described in (Neuhoff et al., 1988). A proteomic analysis by means of mass spectrometry was conducted as described (Pontiggia et al., 2019).

The determination of the protein concentration in the PAMPs fraction was obtained by means of the BRADFORD assay (Bradford, M. M., 1976. A rapid and sensitive method for the quantitation of microgram quantities of protein utilizing the principle of protein-dye binding. Analytical Biochemistry 72, 248-254. https://doi.org/10.1016/0003-2697 (76) 90527-3). 1-5 and 10 μl of PAMPs fraction are resuspended in 0.8 ml of sterile ultrapure H₂O in glass tubes (PYREX® 13×100 mm Disposable Rimless Culture Tubes) to which 0.2 ml of Bradford solution (Bio-Rad Protein Assay Dye Reagent Concentrate, #5000006) is added. The protein concentration is calculated thanks to the comparison with a standard curve prepared with different concentrations of bovine serum albumin (BSA). The absorbance was quantified by means of Varian 50 Scan UV-Visible spectrophotometer at a wavelength of 595 nm.

Arabidopsis thaliana seedlings were grown in liquid medium under specific growth conditions and treated with the PAMPs fraction. Approximately 200 seeds of Arabidopsis thaliana ecotype Columbia-0 (Col-0) were treated with isopropanol for 30 seconds, washed in sterile ultrapure water for 3 minutes under slow stirring with the oscillator (Barnstead Thermolyne M71015 Speci-Mix Test Tube Rocker) and then sterilised with a 1:5 solution of sodium hypochlorite (NaClO) for 5 minutes under slow stirring, followed by 4 washes in sterile ultrapure water. The seeds were stored in the dark at 4° C. for three days for germination stratification. For the growth in liquid medium, the seeds were germinated in 6-well plates (VWR plate tissue culture. Catalogue number: 10062-892) (approximately 10 seeds/well) containing 3 mL of liquid MS/2 medium (2.2 g/l of Murashige and Skoog medium from Duchefa Biochemie, M0221.0050+0.5% sucrose, pH 5.7) per well. The plates were incubated in a controlled environment growth chamber maintained at 22° C. with a 16 h light/8 h dark cycle and a light intensity of 120 mmol m⁻² s⁻¹. After 8 days of growth, the seedlings (10 per well) were transferred into 12-well multiwells (Primo® Multiwell Plate 12 wells, ET3012) containing 1 ml MS/2. At 48 h after transfer, 10-day-old Arabidopsis seedlings were treated with distilled water or PAMPs fraction (1 μg/ml) for 5, 10 and 20 minutes. The protein extraction and detection of phosphorylations of activated MAPKs using the Phospho-p44/42 MAPK (Erk1/2) (Thr202/Tyr204) antibody (Cell Signaling Technology, https://www.cellsignal.com/) were performed as described in (Rebaque et al., 2021).

To assess the effect of the PAMPs fraction on the expression of defence genes, the 10-day-old Arabidopsis seedlings were treated for 1 h with distilled water or PAMPs fraction (1 μg/ml). The treated samples were taken and stored at −80° C. for the subsequent gene expression analysis.

To determine the effects of the PAMPs fraction on growth, after 7 days of growth in liquid medium, the seedlings (1 per well) were transferred into 12-well multiwells containing 1 ml MS/2. The seedlings were treated with distilled water or different doses of PAMPs fraction (1 and 10 μg/ml) and the fresh weight was measured 7 days after treatment, weighing each individual seedling with an analytical scale.

The adult Arabidopsis plants in pots are grown under specific growth conditions and treated with the PAMPs fraction. The Arabidopsis seeds were sterilised as described above and transferred to petri dishes containing solid MS/2 medium (2.2 g/l MS medium, 18 sucrose, 0.8% agar, pH 5.7). The plates were grown for 7 days under the above-mentioned conditions. Subsequently, the seedlings were transferred to sterile soil and grown in a growth chamber at 22° C. with a cycle of 12 h light and 12 h dark (PAR level of 100 μmol m⁻² s⁻¹). The production of reactive oxygen species (ROS) such as hydrogen peroxide (H₂O₂) was monitored after application of the PAMPs fraction on 4-mm diameter leaf discs obtained from 4-week-old Arabidopsis plants using the luminol assay and a VariosKan Lux luminescence reader (Thermo). The leaf discs were collected from eight different plants, transferred to wells containing water, of a microplate reader, and incubated overnight in the growth chamber where the plants were previously grown. Next, the water was removed, 100 μl reaction buffer (10 μg/ml peroxidase and 20 μM luminol L012) was added per well and the microplate was incubated for 1 hour in the dark. 100 μL of water or the PAMPs fraction (1 μg/ml) was added per well and ROS production was monitored in the microplate reader for 2 hours.

The protective effect of the PAMPs fraction on A. thaliana plants against Botrytis cinerea was evaluated. The necrotrophic fungus B. cinerea strain SF1 (Lionetti, V., Raiola, A., Camardella, L., Giovane, A., Obel, N., Pauly, M., Favaron, F., Cervone, F., Bellincampi, D., 2007. Overexpression of Pectin Methylesterase Inhibitors in Arabidopsis Restricts Fungal Infection by Botrytis cinerea. Plant Physiol 143, 1871-1880. https://doi.org/10.1104/pp.106.090803) was grown for 15 days at 22° C. in the dark on 2% malt extract (Duchefia Biochemie, M1327.0500) with 1.2% micro-agar (Duchefia Biochemie, M1002.1000) and 1% mycological peptone (Duchefia Biochemie, P1328.0500) before spore collection. The spores were collected by wetting the surface of the mycelium with sterile distilled water. The conidia suspension was filtered with sterile gauze to remove the residual mycelium and centrifuged at 3000×g for 3 minutes to discard the supernatant and resuspend the spores in sterile distilled water. The concentration of conidia was determined with the Thoma chamber. 24 h prior to infection with Botrytis, 4-week-old Arabidopsis plants grown in pots under the previously described conditions were sprayed with 2 ml of distilled water and PAMPs fraction (10 μg/ml). The leaves were inoculated by applying 6 drops of 5 μl each of 1×10⁶ conidia/ml of 24 g/l potato dextrose broth (Sigma-Aldrich Potato Dextrose Broth-PDB, P6685-250G) on the leaf surface. The control was inoculated with only PDB (Mock). The infected plants were incubated at 22° C. with a photoperiod of 12 h light and 12 h dark. Some leaves were harvested 8 h after infection and stored at −80° C. for subsequent gene expression analysis. The size of the lesions produced by B. cinerea was assessed as an indicator of susceptibility to the fungus by means of ImageJ 48 h software.

The protective effect of the PAMPs fraction on A. thaliana and tomato (Solanum lycopersicum) plants against Pseudomonas syringae was evaluated. The adult Arabidopsis plants were grown under the conditions described above. The tomato seeds (variety Minibel) were germinated on moist paper overnight and transferred to the soil in a growth room with a 16-hour light/8-hour dark cycle (PAR level of 75 μmol m⁻² s⁻¹) at 23° C. and 35-40% humidity. P. syringae pv. tomato DC3000 bacteria were collected by washing the plate with 2 ml of sterile 10 mM MgCl₂ and the concentration of the bacteria was determined using a VariosKan reader. 4-week-old adult Arabidopsis and tomato plants were sprayed with 2 ml of water, flagellin (1 μM) or PAMPs fraction (10 μg/ml) containing adjuvants such as 0.05% Tween 24 MBAL (Croda, Snaith, UK) for the Arabidopsis pretreatment and 2.5% Tween 24 MBAL and 2.5% UEP-100 (Croda, Snaith, UK) for the tomato pretreatment. After 24 h, the Arabidopsis plants were infected with a bacterial concentration of OD₆₀₀=0.1 added with 0.001% Silwet L-77 surfactant, and the tomato plants were infected with a bacterial concentration of OD₆₀₀=0.5 added with 0.002% Silwet L-77. The induction effect of the defence genes was evaluated in Arabidopsis by harvesting infected leaves at 8 hours after infection. Subsequently, the leaf discs of Arabidopsis and tomato were collected from four different plants at 0 (3 hours) and 3 days after infection and colony-forming units per leaf area were determined as described (Mélida et al., 2020).

Gene expression analysis by means of quantitative PCR was carried out from seedlings and leaves collected from adult plants which were frozen in liquid nitrogen and homogenised with the MM301 mixer (RETSCH) using stainless steel beads (5 mm diameter) for about 1 min at 24 Hz. Total RNA was extracted with the extraction reagent Macherey-Nagel™ NucleoZOL (CAS 108-95-2, 593-84-0). 500 μl of NucleoZOL was used per 50 mg of tissue. 200 μl of nuclease-free water was added to the lysate with NucleoZOL. The sample was vigorously stirred for 15 seconds, incubated at room temperature for 5 minutes and centrifuged for 15 minutes at 12000×g at 4° C. After this step, a semi-solid-pellet containing DNA, proteins and polysaccharides forms at the bottom of the tube and the RNA remains solubilised in the supernatant. Subsequently, the supernatant (500 μl) was transferred into a test tube containing 500 μl of isopropanol and stirred. The samples were incubated at −20° C. for 10 minutes. The samples were centrifuged for 10 minutes at 12000×g at 4° C. The supernatant was removed and discarded. Washings were carried out twice. Specifically, 75% ethanol (500 μl) was added and the sample was centrifuged for 3 minutes at 8000×g at 4° C.; the ethanol was removed from the pellet. The sample was centrifuged again at 8000×g at 4° C. for 3 minutes and left under a hood to allow the residual ethanol to evaporate. Finally, the pellet was dissolved in 50 μl of nuclease-free H₂O. RNA was quantified and 260/280 and 260/230 nm absorbance ratios were measured with Thermo Scientific's NanoDrop. RNA quality was assessed by means of gel electrophoresis on 2% agarose (weight/volume) at 60 V for 25 minutes. 1.7 μl (1:6 dilution) of RNA loading buffer without ethidium bromide was added to 500 ng of RNA and nuclease-free water was added up to a final volume of 10 μl; then the sample was heated at 65° C. for 10 minutes. The gel image was taken with the Gel Doc™ XR+ system. The extracted RNA was treated with DNAse for the digestion of residual DNA in 10 μl of reaction mixture containing 2 μg RNA, 1 μl of 10×RQ1 DNase reaction buffer (PROMEGA), 1 μl of nuclease-free RQ1 DNase reaction buffer (PROMEGA) and nuclease-free H2O. The samples were incubated at 37° C. for 30 minutes to allow the digestion reaction to take place. Then 1 μl of DNase-stopping solution was added and the samples were incubated at 70° C. for 10 minutes to inactivate the DNase.

For the reverse transcription of RNA into cDNA, 15 μl of reaction mixture was prepared containing 4 μl of ImProm. II™ 5× reaction buffer (Promega), 2 μl of MgCl2 (25 mM), 1.25 μl of dNTP (10 mM), 0.4 μl of random primers (Promega), 0.4 μl of oligo (dT) s, 1 μl of ImProm. II™ reverse transcriptase enzyme (Promega), 5 μl of RNA previously treated with DNAse and nuclease-free H2O until the final volume is reached. The samples were incubated for 5 min at 25° C. to allow primer pairing on the sequence, then 1 h at 42° C. to allow the extension reaction and 15 min at 70° C. to inactivate the reverse transcriptase.

Quantitative real-time PCR (qRT-PCR) analysis was performed with the CFX96 Real-Time system (Bio-Rad). A 1 μl of cDNA (corresponding to 50 ng of total RNA) was amplified in 10 μl of reaction mixture containing 1× Go Taq qPCR Master Mix (Promega) and 0.5 μM of each primer. The conditions for amplification were: 95° C. for 2 minutes; 46 amplification cycles: 95° C. for 15 seconds, 58° C. for 15 seconds and a final extension of 72° C. for 15 seconds. The expression levels of each gene, relative to the gene UBQ5 (UBIQUITIN 5), were determined using a modification of the Pfaffl method (Pfaffl, 2001). The primer sequences were generated with the software primer3 (https://primer3.ut.ee/) and are shown in table 1.

| GENE | AGI CODE | FORWARD PRIMER (5′-3′) | REVERSE PRIMER (5′-3′) |
|---|---|---|---|
| CYP81F2 | AT5G57220 | SEQ. ID. NO. 1 | SEQ. ID. NO. 2 |
| | | GTGAAAGCACTAGGCGA | ATCCGTTCCAGCTAGCA |
| | | AGC | TCA |
| FRK1 | AT2G19190 | SEQ. ID. NO. 3 | SEQ. ID. NO. 4 |
| | | TTAAACTCGACGATGCAA | GATGGAAGTTTTCCCGT |
| | | CA | TTT |
| PAD3 | AT3G26830 | SEQ. ID. NO. 5 | SEQ. ID. NO. 6 |
| | | TCGCTGGCATAACACTAT | TTGGGAGCAAGAGTGG |
| | | GG | AGT |
| UBQ5 | AT3G62250 | SEQ. ID. NO. 7 | SEQ. ID. NO. 8 |
| | | CCGTGGTGGTGCTAAGA | AGCTCCACAGGTTGCG |
| | | AGA | TTAG |

Statistical analysis: Student's t-test was used for the statistical analysis. The asterisks indicate statistically significant differences with respect to the untreated plants: three asterisks (***) correspond to the p-value<0.001. The error bars represent the standard deviation or standard error of the mean.

### Results

Analysis of the microbial component of the digestate allowed to identify bacteria belonging to the following orders: Clostridium, Bacillus, Pseudomonas, Paenibacillus, Rubrobacter, Bacteroides, Syntrophosphaera, Thermosynergistes, Chazhemtobacterium, Petrimonas, Acetomicrobium, Limnochordia, Dethiobacter, Mesotoga, Anaerolineae, Fermentimonas, Sedimentibacter, Thermoclostridium A and Sedimentibacter. Archea such as Methanosaeta, Methanoculleus, Methanobacterium and Methanosarcina are also present.

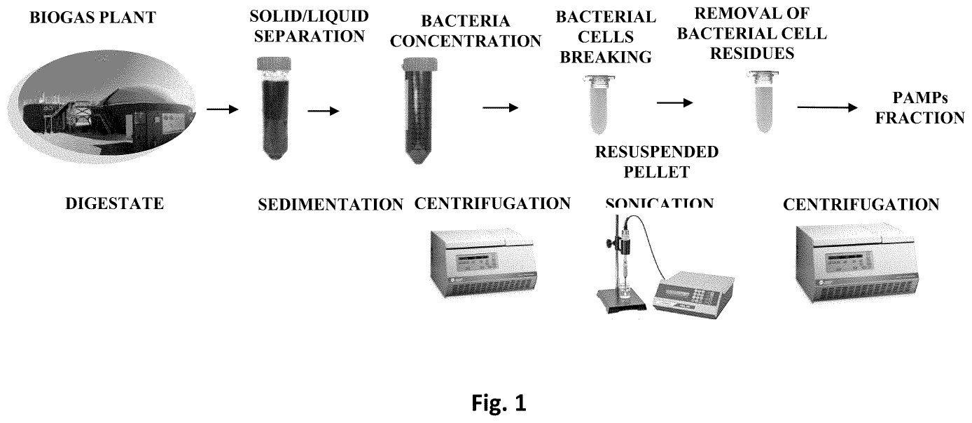

The isolation of the PAMPs fraction occurred using as original feed a digestate from the olive pomace-fueled biogas plant called AGROENERGY managed by the owner AGROLIO SRL, Andria, Bari. The digestate was subjected to a process aimed at isolating proteins, peptides from the microbial population. The digestate (250 ml) was left to settle at +4° C. for 24 h and the liquid fraction (125 ml) was sampled and transferred into 30 ml centrifuge tubes (Nalgene™ Oak Ridge High-Speed PPCO Centrifuge Tubes. Catalogue number: 3119-0030) and centrifuged at 8,000×rpm for 5 minutes to precipitate the microbial portion. After removing the supernatant, the bacterial pellet was resuspended in 5 ml of distilled water. In order to lyse the bacterial cells and solubilise the proteins, peptides and the resulting solution were subjected to high-frequency sound waves for 5 minutes, causing the cellular membranes to break using a sonicator (Sonics vibra-cell VCX 130). The sonication conditions are 70% amplitude with pulser function 20 seconds ON and 20 seconds OFF. The sonicated solution was then subjected to centrifugation at 15,000×g for 30 minutes at 4° C. The cell residue was discarded, while 4 ml of supernatant was collected and aliquoted into new tubes (FIG. 1). The solution obtained, defined PAMPs fraction, has a protein concentration of 173.8 μg/ml. This fraction will be called the PAMPs fraction and will be used for the subsequent experiments. In parallel, pretreatment tests were carried out with pasteurisation of the microbial fraction at 85° C. for 5 min, obtaining similar results to the sonication.

The proteins contained in the PAMPs fraction were separated according to their size by SDS-PAGE and stained through Coomassie. Predominant protein bands of 130 kDa, 65 kDa, 40 kDa, 30 kDa and other less intense bands were detected. The protein composition was then assessed, following extraction of the bands from gels, by means of proteomic analysis through mass spectrometry. The analysis revealed the presence of the proteins flagellin and elongation factor, of which certain fragments (i.e., Flg22 and elf18, respectively) are immunogenic when administered to plants.

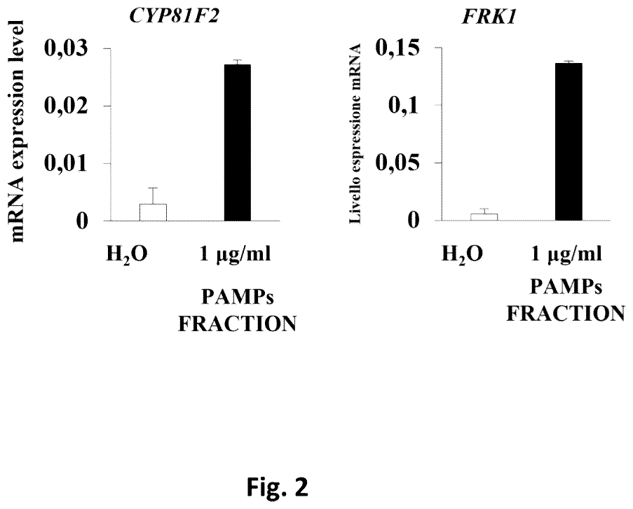

It was shown that the treatment with PAMPs fraction is capable of eliciting defence responses in plants. In fact, the concentration of 1 μg/ml induces ROS production with respect to the treatment with water. Subsequently, the ability of the PAMPs fraction to induce phosphorylation activation of MPK6/MPK3/MPK4/MPK11 was monitored, proving capable of activating the phosphorylation of MAPK6 and MAPK3 already after 5 min and the phosphorylation of MAPK4/11 20 min after treatment with respect to the treatment with water where no phosphorylation activation was observed. The immunostimulating effect of the PAMPs fraction was also evaluated by analysing the expression of defence genes following treatment of Arabidopsis seedlings grown in liquid medium for 1 hour with 1 μg/ml PAMPs fraction or with water. The CYP81F2 (CYTOCHROME P450, FAMILY 81) and FRK1 (FLG22-INDUCED RECEPTOR-LIKE KINASE 1) genes were selected for these experiments. CYP81F2 encodes for a biosynthetic enzyme involved in the accumulation of an Indole Glucosinolate (4-methoxy-indol-3-ylmethyl Glucosinolate), which is essential for Arabidopsis defence responses against several pathogens including the phytopathogenic fungus Botrytis cinerea (AbuQamar et al., 2017; Clay et al., 2009). FRK1 (FLG22-INDUCED RECEPTOR-LIKE KINASE 1) encodes for a kinase receptor with leucine-rich repetitions involved in the activation of defence signalling pathways following the recognition of bacterial flagellin (FLG22) (Giovannoni et al., 2021). The expression of both CYP81F2 and FRK1 is induced in Arabidopsis seedlings following treatment with PAMPs fraction, unlike the water treatment where no induction is observed (FIG. 2).

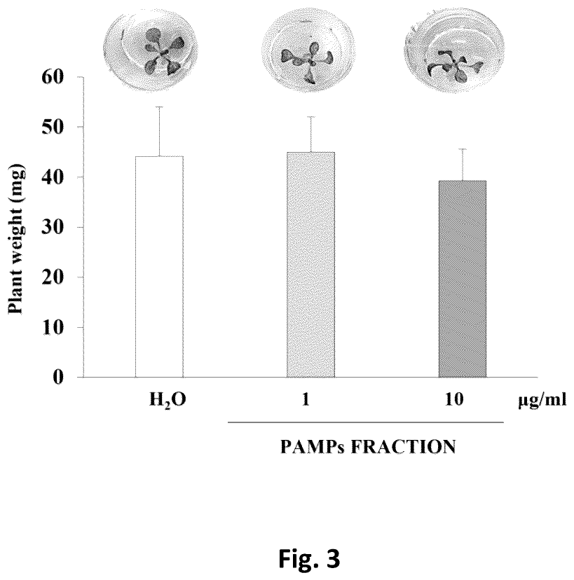

The effect of the PAMPs fraction on plant growth was tested on Arabidopsis seedlings grown in liquid medium both in the absence and presence of different concentrations of the PAMPs fraction (1 and 10 μg/ml). The fresh weight of the seedlings was measured 7 days after treatment. No significant difference was observed following treatment with 1 μg/ml, the concentration previously used to test the induction of defence responses, and even with a 10-fold higher concentration (10 μg/ml), indicating that appropriate concentrations of the PAMPs fraction can be used without adversely affecting plant growth (FIG. 3).

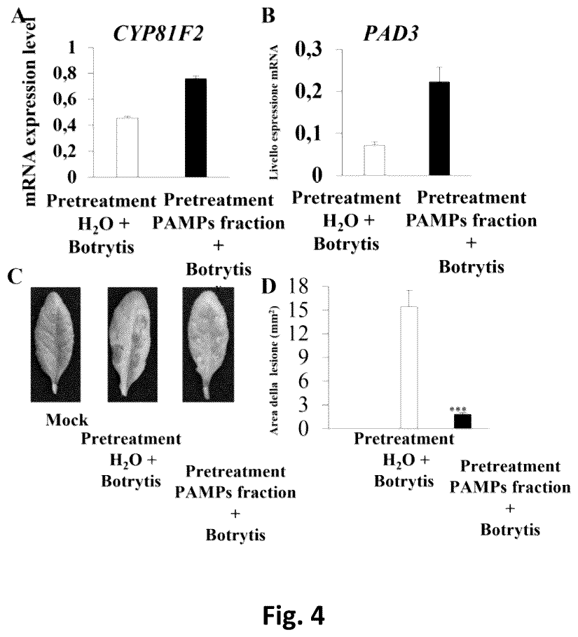

Pretreatment with the PAMPs fraction induces the resistance of Arabidopsis against the necrotrophic fungus B. cinerea. To assess the protective effect against B. cinerea, 4-week-old Arabidopsis plants were sprayed with 2 ml of PAMPs fraction at 10 μg/ml. After 24 h, the leaves were inoculated with the necrotrophic fungus B. cinerea and the expression of the CYP81F2 and PAD3 (PHYTOALEXIN DEFICIENT 3) genes was analysed 8 h after infection. PAD3 encodes the enzyme involved in the last step of camalexin biosynthesis and has been reported to be necessary for resistance against B. cinerea (Ferrari et al., 2007; Giovannoni et al., 2021; Gravino et al., 2015). The plants pre-treated with the PAMPs fraction showed a higher expression of the defence genes with respect to the non-pretreated plants (FIGS. 4A and B). To assess how effective the induction of immunity by the PAMPs fraction can be in counteracting fungal infection, resistance to B. cinerea was determined by measuring the lesion area on leaves 48 h after infection. The fungus-related symptom is significantly reduced in the leaves of plants pre-treated with the PAMPs fraction with respect to the plants pre-treated with water (FIGS. 4C and D). In particular, the lesion is smaller and dehydrated in the immunostimulated leaves, indicating a more restriction of fungal spread limited by the plant's hypersensitive response. This observation is confirmed by the quantitative measurement of the lesion in the leaves pre-treated with PAMPs fraction with respect to those pre-treated with water. The data clearly indicate how the pretreatment with the PAMPs fraction, by pre-inducing an immune response, helps the plant to limit the subsequent phytopathogenic infection.

It has been demonstrated that the PAMPs fraction also induces protection of adult Arabidopsis plants against the hemibiotrophic bacteria P. syringae pv. tomato DC3000. Adult Arabidopsis plants (4 weeks old) were pre-treated with the PAMPs fraction (10 μg/ml) 24 hours before infecting them with P. syringae. At 8 hours post-infection, the plants pre-treated with the PAMPs fraction showed a higher induction of CYP81F2 and WRKY53 expression with respect to those pre-treated with water, and bacterial growth was significantly reduced at 3 days post-infection with respect to the plants pre-treated with water.

Similar results were also found in the adult tomato plant. Tomato plants treated with the PAMPS fraction exhibited increased ROS production and reduced bacterial population when infected with P. syringae. All these results indicate that a preventive pretreatment with the PAMPs fraction can improve the immune performance and protection of plants against plant pathogens.

1. A process for the treatment of a digestate derived from an anaerobic digestion of an olive pomace comprising the following steps:
(a) undergoing or subjecting the digestate derived from the anaerobic digestion of the olive pomace to sedimentation to obtain a liquid fraction and a solid fraction;
(b) sampling the liquid fraction obtained at the end of step a) and centrifugation of the liquid fraction to obtain a liquid supernatant fraction and a solid pellet of bacterial origin;
(c) resuspending the solid pellet of bacterial origin as obtained at the end of step b);
(d) undergoing the resuspended solid pellet of bacterial origin as obtained at the end of step c) to mechanical cellular breaking by sonication to obtain a solution; and
(e) additional centrifugation of the solution obtained at the end of step d) to obtain a cellular residue fraction and a liquid phase as final product,
wherein the final product is a not-infectious, bacterial-protein and bacterial-peptides enriched solution.

2. A not-infectious, bacterial-protein and bacterial-peptides enriched solution obtained by a process comprising the following steps:
(a) sedimentation of a digestate derived from an anaerobic digestion of an olive pomace to obtain a liquid fraction and a solid fraction;
(b) sampling the liquid fraction obtained at the end of step a) and centrifugation of the liquid fraction to obtain a liquid supernatant fraction and a solid pellet fraction, wherein the pellet is of bacterial origin;
(c) resuspending the solid pellet of bacterial origin as obtained at the end of step b);
(d) undergoing the resuspended solid pellet of bacterial origin as obtained at the end of step c) to mechanical cellular breaking by sonication to obtain a solution; and
(e) additional centrifugation of the solution obtained at the end of step d) to obtain a cellular residue fraction and a liquid phase being the solution as final product.

3. A method for immunostimulating a plant immune system comprising administering to the plant the final product the solution as obtained from the process of claim 1.

4. The process of claim 1, wherein the solid pellet of bacterial origin comprises one or more bacterial species belonging to the phyla selected from the group consisting of: Acidobacteria, Actinobacteria, Bacteroids, Chloroflexi, Euryarcheota, Firmicutes, Proteobacteria, Spirochetes, Thermotogae, Tenericutes and Verrucomicrobes.

5. The process of claim 7, wherein the bacterial protein PAMPS fraction is in an amount ranging from 10 to 500 μg/ml.

6. The process of claim 1, wherein the solution comprises mixture of bacterial proteins, peptides, and lipopolysaccharides with synergistic PAMP activity.

7. The process of claim 1, wherein the final product is a not-infectious, bacterial-protein and bacterial-peptides enriched solution comprising a bacterial-derived lipopolysaccharides with PAMP activity.

8. The solution of claim 2, wherein the not-infectious, bacterial-protein and bacterial-peptides enriched solution comprises bacterial-derived lipopolysaccharides with PAMP activity.

9. The solution of claim 2, wherein the solid pellet of bacterial origin comprises one or more bacterial species belonging to the phyla selected from the group consisting of: Acidobacteria, Actinobacteria, Bacteroids, Chloroflexi, Euryarcheota, Firmicutes, Proteobacteria, Spirochetes, Thermotogae, Tenericutes and Verrucomicrobes.

10. A method for immunostimulating a plant immune system comprising administering to the plant the solution of claim 2.


SEQUENCE LISTING (8 sequences)

SEQ ID NO: 1 - DNA, 20 bp, synthetic construct
  source 1..20: mol_type = other DNA
gtgaaagcactaggcgaagc

SEQ ID NO: 2 - DNA, 20 bp, synthetic construct
  source 1..20: mol_type = other DNA
atccgttccagctagcatca

SEQ ID NO: 3 - DNA, 20 bp, synthetic construct
  source 1..20: mol_type = other DNA
ttaaactcgacgatgcaaca

SEQ ID NO: 4 - DNA, 20 bp, synthetic construct
  source 1..20: mol_type = other DNA
gatggaagttttcccgtttt

SEQ ID NO: 5 - DNA, 20 bp, synthetic construct
  source 1..20: mol_type = other DNA
tcgctggcataacactatgg

SEQ ID NO: 6 - DNA, 19 bp, synthetic construct
  source 1..19: mol_type = other DNA
ttgggagcaagagtggagt

SEQ ID NO: 7 - DNA, 20 bp, synthetic construct
  source 1..20: mol_type = other DNA
ccgtggtggtgctaagaaga

SEQ ID NO: 8 - DNA, 20 bp, synthetic construct
  source 1..20: mol_type = other DNA
agctccacaggttgcgttag